(19) United States
(12) Patent Application Publication
(10) Pub. No.: US 2026/0269044 A1
(43) Pub. Date: Sep. 10, 2026

(54) METHOD AND APPARATUS FOR WELLNESS TRAINING

(71) Applicant: DENDRO TECHNOLOGIES INC., NEW YORK, NY (US)

(72) Inventors: Moshe Iko HASON, TEL-AVIV (IL); Adi WALLACH, NEW YORK, NY (US)

(21) Appl. No.: 19/480,001

(22) PCT Filed: Jun. 19, 2024
(86) PCT No.: PCT/IB2024/055977

§ 371 Date: Oct. 29, 2025

Related U.S. Application Data
(60) Provisional application No. 63/521,730, filed on Jun. 19, 2023.

(51) Int. Cl. G16H 20/30; A63F 13/212

(52) U.S. Cl. CPC G16H 20/30; A63F 13/212

(57) ABSTRACT

Disclosed are methods and apparatus for encouraging a user to use a wellness device, preferably often and even regularly, and/or to use a wellness device correctly. Specifically disclosed is, a wellness device which, with often and/or regular use, may increase at least one parameter of the well-being of the user, preferably the physical and/or emotional well-being of the user. In some embodiments, the wellness device is a breathing biofeedback device.

### CROSS-REFERENCE TO RELATED APPLICATION

The present application is a National Phase entry under 35 U.S.C. § 371 of International Application No. PCT/IB2024/055977, which claims priority from US Provisional Patent Application 63/521,730, filed 19 Jun. 2023, which is hereby incorporated by reference as if fully set-forth herein.

### FIELD

The present disclosure relates to the field of training, and more particularly, but not exclusively, to methods and apparatus suitable for encouraging a user to use a device such as a breathing biofeedback device.

### BACKGROUND

Breathing is the process by which animals take in oxygen necessary for cellular metabolism and release the carbon dioxide that accumulates in the body as a result of the expenditure of energy.

Breathing can be affected by conditions such as emotional state (e.g., stress, excitement, fear, etc.), physical exercise, temperature, ingested or inhaled substances, weather and environmental conditions, various diseases and obesity.

Breathing is one of the few bodily functions that can be controlled, within limits, both consciously and unconsciously. Conscious control of breathing is provided by the autonomic nervous system via the cerebral cortex. Conscious control of breathing is common in many forms of meditation, such as forms of yoga, and controlled deep breathing has been found to induce relaxation, leading to lowered blood pressure and heart rate, reduced amounts of stress hormones, reduced lactic acid build-up in muscle tissue, balanced levels of oxygen and carbon dioxide in the blood, improved immune system functioning, increased physical energy and feelings of calm and well-being. The use of slow, deep breathing, as a means of promoting relaxation is considered to be of help in managing stress, as well as a range of disorders which are related to, or affected by stress including anxiety, asthma, chronic fatigue syndrome, chronic pain, high blood pressure, insomnia, panic attacks, labor pain, and some skin conditions such as eczema, and as well as various psychosomatic and psychoneurotic disorders.

Various biofeedback techniques and devices that assist a user in achieving controlled breathing are known.

Particularly suitable breathing biofeedback devices for assisting a user to achieve controlled breathing are described in U.S. Pat. No. 11,179,101 and patent application publication WO 2023/081489, both by the Inventor, or the commercially-available CalmiGo® device from Dendro Technologies, Inc. (New York, NY, USA).

Breathing biofeedback devices may be used in acute situations. For example, a person uses a breathing biofeedback device to relieve an incidental episode of shortness of breath, for instance, an episode of shortness of breath associated with stress.

It has been found that if a user uses a breathing biofeedback device often, and, preferably even regularly, even when there is no acute reason to do so, the user may learn to habitually breathe in a controlled, and therefore improved, way, for example deeper breathing with a relatively long exhalation duration. Further, the user may learn to easily recall how to achieve such controlled breathing in a future acute situation.

It would be useful to have methods and/or apparatus suitable for encouraging a user to use a wellness device such as a breathing biofeedback device more often and even regularly.

### SUMMARY

Some embodiments of the invention relate to methods and apparatus suitable for encouraging a user to use a wellness device preferably often and even regularly. In some embodiments, the wellness device is a wellness device which often and/or regular use may increase at least one parameter of the well-being of the user, preferably the physical and/or emotional well-being of the user. For example, in some embodiments the wellness device is a breathing biofeedback device which often and/or regular use may teach a user to habitually breathe in a controlled way. Habitual controlled breathing typically increases both the physical and emotional well-being of a user.

According to the method of the teachings herein, there is provided a method for encouraging a user to use a wellness device, comprising:
providing the user with a video game program to play on a display device;
providing information about the use of a wellness device by the user as input for the video game program; and
based on the provided information, the video game program improving the in-game experience of the user.

The method can be considered as using the improved in-game experience as a highly-effective reward to motivate a user to use the wellness device a. more often and/or b. more regularly and/or c. more correctly. As a result of one or more a, b and c, the user experiences an increase of the positive effects of the use of the device. Further, in some embodiments, as a result of one or more of a, b and c, the user adopts the behaviors imposed by the use of the device (e.g., correct breathing) as second-nature or the user learns to recall the behaviors imposed by the use of device more easily.

In some embodiments, as a consequence of the improved in-game experience of the user, the user uses the wellness device more often (i.e., more frequently) than without such an improved in-game experience. By using the wellness device more often, the user experiences an increase of the positive effects of the use of the device.

Additionally or alternatively, in some embodiments, as a consequence of the improved in-game experience of the user, the user uses the wellness device regularly. By using the wellness device more regularly, the user is “trained” to include the use of the device in the user's life, e.g., in the user's daily regimen, so the user experiences an increase of the positive effects of the use of the device.

Additionally or alternatively, in some embodiments, as a consequence of the improved in-game experience of the user, the user uses the wellness device more correctly. All other things being equal, a more correct use of the device increases the positive effects of the use of the device.

In some embodiments, a result of implementing the method is that at least one parameter of the well-being of the user is increased. In some embodiments, a result of implementing the method is that the physical well-being of the user increases and/or that the emotional well-being of the user increases.

The display device is any suitable device on which a video game program can be played. In some embodiments, the display device is a device selected from the group consisting of a laptop computer, a desktop computer, a tablet and a smartphone.

The wellness device is any suitable wellness device. In some preferred embodiments, the wellness device is a breathing biofeedback device. In some such embodiments, by using the method the user is taught to habitually breathe in a controlled way. In some such embodiments, by using the method the user is taught to recall how to breathe in a controlled way.

The video game program is any suitable video game. In preferred embodiments, the video game program is a game comprising an in-game base. In some such embodiments, the video game program is a game comprising an in-game base selected from the group consisting of a world-building game, a sandbox game, a real-time strategy game, a role-playing game, a simulation game and an action-adventure game.

In some embodiments, the improving of the in-game experience comprises rewarding the user for using the wellness device often and/or correctly. In some embodiments improving the in-game experience comprises providing a reward for use of the wellness device. Additionally or alternatively, in some embodiments improving the in-game experience comprises providing a reward for correct use of the wellness device.

In some embodiments, the value of the provided reward is related to a frequency of use of the wellness device. For example, in some embodiments the value of the provided reward increases for each day that the wellness device is used. In some embodiments, the value of the provided reward is related to the number of consecutive days that the wellness device is used. In some embodiments, the value of the provided reward is related to the degree of conformity and/or compliance with a pre-determined schedule that the wellness device is used.

In some embodiments, at least some of the rewards are in-game rewards. In some such embodiments, at least one in-game reward is selected from the group of in-game rewards consisting of:

an in-game reward comprising allowing the user to play the video game program more often and/or for a longer duration;
an in-game reward comprising an in-game encounter;
an in-game reward comprising information useful for playing the game;
an in-game reward comprising provision of an in-game item;
an in-game reward comprising provision of increased in-game storage capacity;
an in-game reward comprising an increased level of a player character;
an in-game reward comprising an increased number of hit points of a player character; and
an in-game reward comprising improvement of a player's in-game base.

In some embodiments, at least some of the rewards are tangible rewards. In some such embodiments, wherein at least one tangible reward is selected from the group of tangible rewards consisting of: a coupon, a gift card and a subscription.

In some embodiments, the information provided about the use of the wellness device relates to use of the wellness device when the video game program is not running.

Additionally or alternatively, in some embodiments, the information provided about the use of the wellness device relates to use of the wellness device when the video game program is running.

Additionally or alternatively, in some embodiments, the information provided about the use of the wellness device relates to use of the wellness device while the video game program is played.

According to an aspect of some embodiments of the teachings herein, there is also provided an apparatus suitable for encouraging a user to use a wellness device, comprising:
a display device having a computer processor functionally associated with a display, the computer processor configured:
   a. to run at least part of a video game program, and
   b. using the display to display game screens to a user playing the video game program,
the display device further configured to:
   receive information from a wellness device about the use of the wellness device; and
   provide the received information to the video game program.

In some embodiments, the device is configured to implement one or more of the embodiments of the method according to the teachings herein.

The video game program is configured to use the information received about the use of wellness device to improve the in-game experience. For example, and as detailed below, in some embodiments the video game is configured to reward the user for using the wellness device often and/or more correctly.

### Display Device and Display

The display is any suitable display. In preferred embodiments, the display is a component of the display device. In some embodiments, the display is selected from the group consisting of an LCD screen, a plasma screen, a LED screen and an OLED screen.

The display device is any suitable computer display device. In some embodiments, the display device is selected from the group consisting of a laptop computer, desktop computer, tablet and smartphone.

In some embodiments, the configuration of the display device to receive the information from the wellness device about the use of the wellness device is through wireless communication, e.g., Bluetooth®, Wi-Fi, NFC. Such configuration includes both hardware (e.g., a wireless transceiver) and the required software/firmware to use the hardware.

In some embodiments, the configuration of the display device to receive the information from the wellness device about the use of the wellness device is through wired communication, e.g., USB-C.

In some embodiments, the display device is configured to provide the received information about the use of the wellness device to the video game program when the video game program is being played on the display device.

In some embodiments, the display device is configured to provide the received information about the use of the wellness device to the video game program only when the video game program is being played on the display device.

In some embodiments, the display device is configured to provide the received information about the use of the wellness device to the video game program when the video game program is running on the display device, even if running in the background and not being played on the display device.

In preferred embodiments, the display device is configured to receive the information about the use of the wellness device and, if the video game program is not being played on the display device and/or is not running on the display device, the display device is configured to store the received information and to provide the stored information to the video game program at a later time. Typically, “the later time” is when the video game program is turned on the display device or when the video game program is played on the display device. Such embodiments are preferred as these allow a person to use the wellness device independently of playing the game and still to gain the advantages of the teachings herein. In some embodiments, the received information that is stored, is stored in a computer memory. In some embodiments, the computer memory is computer memory of a device selected from the group consisting of the wellness device, the display device, a cloud and a server.

An embodiment of the apparatus is depicted in FIG. 1 where apparatus 2 comprises a display device 4 (a general-purpose smartphone) having a display 6 (the touchscreen of display device 4). As with known smartphones, display device 4 includes software and hardware such as a computer processor, computer memory and is configured for both wired (through a USB port) and wireless communication (Bluetooth®, Wi-Fi and NFC).

Installed on display device 4 is a video game program which is configured to run on display device 4 in the usual way, including using display 6 to display game screens to a user playing the video game program. As known in the art of video gaming, a “game screen” is the information (including drawings, pictures, figures, texts and/or images) that the video game program displays on a display screen to be seen by a user (the person playing the game). The installed video game program is configured to receive user-input such as game commands from the input component of display device 4 (since display device 4 is a smartphone, the input component is the touchscreen, display 6) and optionally other display device components, for example, a smartphone accelerometer.

In some embodiments, all of the video game program runs on display device 4, that is to say the video game program is configured to run entirely on display device 4. In alternative embodiments, only a part of the video game program runs on display device 4, that is to say, the video game program is configured that part of the video game program runs on the display device and a part of the video game program runs on a remote computer, similar or analogous to known video game programs such as Pokemon Go® or MMORPG games.

### Wellness Device

The wellness device is any suitable well device. In some embodiments, the wellness device is a breathing biofeedback device. Exemplary suitable breathing biofeedback devices are appropriately-configured breathing biofeedback devices such as described in U.S. Pat. No. 11,179,101 and patent application US Ser. No. 18/703,345 published as WO 2023/081489, both by the Inventor, or the commercially-available CalmiGo® device from Dendro Technologies, Inc. (New York, NY, USA).

In FIG. 1, display device 4 is configured to receive information from a wellness device 8 about the use of the wellness device and to provide the received information to the video game program. In FIG. 1, wellness device 8 is a breathing biofeedback device such as described in U.S. Ser. No. 18/703,345.

In some embodiments, the wellness device is not a component of the apparatus. In such embodiments, in FIG. 1 wellness device 8 is not a component of apparatus 2.

Alternatively, in some embodiments, the apparatus further comprises a wellness device. In such embodiments, in FIG. 1 wellness device 8 is a component of apparatus 2.

In some embodiments, the display device is configured to receive the information about the use of the wellness device through wireless communication and the wellness device is configured to transmit the information to the display device through wireless communication. In FIG. 1, wellness device 8 comprises a Bluetooth® wireless communication module allowing a computer controller of wellness device 8 to wirelessly transmit information about the use of wellness device 8 to display device 4.

In some embodiments, the display device is configured to receive the information about the use of the wellness device through wired communication and the wellness device is configured to transmit the information to the display device through wired communication. To depict such an embodiment in FIG. 1, a communication cable such as a USB cable would be connecting wellness device 8 with display device 4.

As noted above, the video game program is configured to use the information received about the use of wellness device to improve the in-game experience.

In some embodiments, the video game program is configured to provide rewards for use of the wellness device as at least part of the improved in-game experience.

In some embodiments, the video game program is configured to provide rewards for correct use of the wellness device, as opposed to less correct or incorrect use of the wellness device.

In some embodiments, the video game program is configured so that the value of the provided rewards is related to a frequency of use of the wellness device. In some such embodiments, the video game program is configured so that a reward is provided for each day that the wellness device is used. In some such embodiments, the video game program is configured so that a reward is provided for each consecutive and/or number of consecutive days that the wellness device is used. For example, in some such embodiments, the video game program is configured so that consecutive daily use for an entire week provides a substantially more valuable reward.

In some embodiments, the video game program is configured so that the value of the provided rewards is related to the degree of conformity and/or compliance with a pre-determined schedule that the wellness device is used.

In some embodiments, the video game program is configured to provide rewards for use of the wellness device even when the video game program is not running on the display device. In some embodiments, the video game program is configured to provide rewards only when the wellness device is used while the game program is running on the display device.

In some embodiments, the video game program is configured to provide rewards only when the wellness device is used while the video game program is running and being played on the display device.

In some preferred embodiments, the video game program is a game comprising an in-game base. In some such embodiments, the video game program is a game comprising an in-game base selected from the group consisting of a world-building game, a sandbox game, a real-time strategy game, a role-playing game, a simulation game and an action-adventure game.

In some embodiments, at least some of the rewards are in-game rewards. In some such embodiments, wherein at least one in-game reward is selected from the group of in-game rewards consisting of:
an in-game reward comprising allowing the user to play the video game program more often and/or for a longer duration;
an in-game reward comprising an in-game encounter;
an in-game reward comprising information useful for playing the game;
an in-game reward comprising provision of an in-game item;
an in-game reward comprising provision of increased in-game storage capacity;
an in-game reward comprising an increased level of a player character;
an in-game reward comprising an increased number of hit points of a player character; and
an in-game reward comprising improvement of a player's in-game base.

In some embodiments, at least some of the rewards are tangible rewards. In some such embodiments, wherein at least one tangible reward is selected from the group of tangible rewards consisting of: a coupon, a gift card and a subscription.

Additional aspects and embodiments of the invention are described in the description hereinbelow and in the appended claims and Figures.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 schematically depicts an embodiment of an apparatus according to the teachings herein: and

FIGS. 2A, 2B, 2C and 2D schematically depict exemplary screenshots of a display component of a apparatus according to the teachings herein.

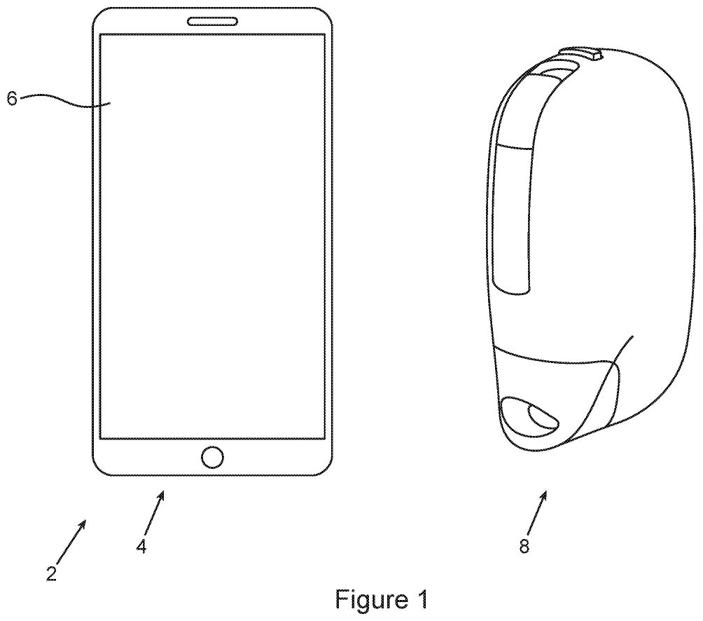

### DETAILED DESCRIPTION

Unless otherwise defined, all technical and scientific terms used herein have the same meaning as commonly understood by one of ordinary skill in the art to which the invention pertains. In case of conflict, the specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof. As used herein, the indefinite articles “a” and “an” mean “at least one” or “one or more” unless the context clearly dictates otherwise.

As used herein, when a numerical value is preceded by the term “about”, the term “about” is intended to indicate +/−10%. As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Embodiments of methods and/or devices described herein may involve performing or completing selected tasks manually, automatically, or a combination thereof. Some methods and/or devices described herein are implemented with the use of components that comprise hardware, software, firmware or combinations thereof. In some embodiments, some components are general-purpose components such as general-purpose computers or digital processors. In some embodiments, some components are dedicated or custom components such as electronic circuits, integrated circuits or software.

For example, in some embodiments, some of an embodiment is implemented as a plurality of software instructions executed by a data processor, for example which is part of a general-purpose or custom computer. In some embodiments, the data processor or computer comprises volatile memory for storing instructions and/or data and/or a non-volatile storage, for example, a magnetic hard-disk and/or removable media, for storing instructions and/or data. In some embodiments, implementation includes a network connection. In some embodiments, implementation includes a user interface, generally comprising one or more input devices (e.g., allowing input of commands and/or parameters) and output devices (e.g., allowing reporting parameters of operation and results).

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

The invention, in some embodiments thereof, relates to methods and apparatus suitable for encouraging a user to use a wellness device preferably often and even regularly, in some embodiments, the wellness device being a device which often and/or regular use may increase at least one parameter of the well-being of the user, preferably the physical and/or emotional well-being of the user. For example, in some embodiments the wellness device is a breathing biofeedback device which often and/or regular use may teach a user to habitually breathe in a controlled way.

The principles, uses and implementations of the teachings herein may be better understood with reference to the accompanying description and figures. Upon perusal of the description and figures present herein, one skilled in the art is able to implement the invention without undue effort or experimentation. In the figures, like reference numerals refer to like parts throughout.

Before explaining at least one embodiment in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

### Apparatus of the Teachings Herein

In some embodiments, an apparatus of the teachings herein comprises a display device having a computer processor functionally associated with a display (e.g., an LCD or LED screen), the computer processor configured to run at least part of a video game program (herein also called a game or a video game) and using the display to display game screens to a user playing the game. The display device is further configured to receive (wired (e.g., USB-C) and/or wirelessly (e.g., Bluetooth®, Wi-Fi, NFC)), information from a wellness device about the use of the wellness device which information is provided to the video game program.

In some embodiments, the apparatus comprises the display device but does not comprise the wellness device. In some embodiments, the apparatus comprises both the display device and the wellness device.

In some embodiments, the apparatus is configured to receive and provide the information about the use of the wellness device to the video game program when the video game program is being played. In some embodiments, the apparatus is configured to receive and provide the information about the use of the wellness device to the video game program only when the video game program is being played.

Additionally or alternatively, in some embodiments, the apparatus is configured to receive and provide the information about the use of the wellness device to the video game program when the video game program is running on the display device, even if the game is running in the background of the display device and not being played.

Additionally or alternatively, in some embodiments, the apparatus is configured to receive the information about the use of the wellness device and, if the video game is not being played and/or not running, the information is stored (e.g., in a computer memory of the wellness device, of the display device, or any other suitable memory, for example a cloud or server) and provided to the video game at a later time.

The computer processor and the display of the display device are any suitable computer processor and display. For example, in some embodiments the display device is a known laptop computer, desktop computer, tablet or smartphone that comprises software and/or hardware and/or firmware to implement the teachings herein.

The wellness device is any suitable wellness device. In some embodiments, the wellness device is a wellness device which often and/or regular use may increase at least one parameter of the well-being of the user, for example the physical and/or emotional well-being of the user. In addition to the wellness functionality of the wellness device, the wellness device is configured to provide, wired (e.g., through a wired USB-C connection) or wirelessly (e.g., Bluetooth®, Wi-Fi, NFC), information about the use of the wellness device to the video game program of the display device.

In some embodiments, the wellness device is a breathing biofeedback device which often and/or regular use may teach a user to habitually breathe in a controlled way. Habitual controlled breathing increases the physical and emotional well-being of the user. Particularly suitable breathing biofeedback devices include devices described in U.S. Pat. No. 11,179,101, in patent application PCT/US2022/049182 and the commercially-available CalmiGo® device from Dendro Technologies, Inc. which have been appropriately modified to implement the teachings herein.

The computer processor of the display device is configured to run at least part of the video game program. In some embodiments, the computer processor of the display device runs all of the game locally. In some embodiments, the computer processor of the display device runs only a portion of the game and another portion of the game is run on a remote computer processor, in a manner analogous to Pokémon GO by Niantic, Inc. (San Francisco, California, USA).

The video game program is any suitable video game program which is configured to provide rewards for use, especially correct use, of the wellness device. In some embodiments, some or all of the rewards are in-game rewards. Additionally or alternatively, in some embodiments some of the rewards are tangible rewards, e.g., a coupon, a subscription, a gift card. A tangible reward is a reward which exists outside of the video game program.

In some embodiments the value of rewards that are provided to a user is related to frequency of use of the wellness device, for example, the more the wellness device is used the more valuable the rewards.

Additionally or alternatively, in some embodiments the value of rewards is related to the degree of conformity and/or compliance with a predetermined schedule that the wellness device is used, i.e., the user is provided with a schedule for use (e.g., including frequency and duration of use sessions) and the closer the user follows the schedule the more valuable the rewards. For example, in some embodiments each day of use of the wellness device will provide the user with a reward and, additionally, consecutive daily use for an entire week provides a substantially more valuable reward for the user.

Additionally or alternatively, in some embodiments the value of rewards is related to the degree of conformity with a predetermined manner in which the wellness device is used. For example, when the wellness device is a breathing biofeedback device which assists in achieving controlled breathing, the closer the actual breathing of the user is to a desired breathing regimen, the more valuable the reward.

In some embodiments, use of the wellness device provides rewards even when the game program is not running.

In some embodiments, rewards are provided only when the wellness device is used while the game program is running (being played or is running in the background of the display device).

In some embodiments, rewards are provided only when the wellness device is used while the game program is being played on the display device.

### Video Game Program

The video game program is any video game which is configured to provide rewards. As noted, rewards are in-game rewards and/or tangible rewards.

For example, in some embodiments, an in-game reward is allowing the user to play the video game more often and/or for a longer duration. For example, if the person used the wellness device for up to 15 minutes they are allowed to play the game for 10 minutes, up to 20 minutes they are allowed to play for 20 minutes but, if the person used the wellness device for more than 30 minutes, they are allowed to play the entire day.

For example, in some embodiments, an in-game reward is an in-game encounter (e.g., with a non-player character, a puzzle, a metagame, a new game level, additional content) which is otherwise of limited availability or unavailable. For example, in some embodiments, an in-game reward is that a new game level is made accessible for play.

For example, in some embodiments, an in-game reward is information useful for playing the game, for example a clue to solve a puzzle.

For example, in some embodiments, an in-game reward is provision of an in-game item such as in-game money, coin, tool, weapon, food, fuel or commodity which is otherwise of limited availability or unavailable. In some such embodiments, an in-game reward is increased storage capacity, e.g., of items and/or money and/or coins.

For example, in some embodiments, an in-game reward is provision of an in-game improvement of a player character for example, an increased player character level or hit points.

The person who desires to advance and/or do well in the game uses the wellness device in order to receive the in-game rewards. As a result, and depending on the embodiment, the person uses the wellness device more often and/or more correctly, thereby improving the wellness of the person.

In some preferred embodiments, the video game is a game where the player has an in-game base (for example, some embodiments of world-building game, a sandbox game, a real-time strategy game, a role-playing game, a simulation game, an action-adventure game) being a virtual location to which the player can return during play, and an in-game reward comprises improvement of the base, for example, increased base size, increased base quality, additional items/decorations located in the base. Typical such embodiments include a base which is a house, a zoo, a farm, a garden and a museum. In-game rewards can include one or more of a virtual furnishing, decoration, additional and/or improved construction material, tool, device, animal, plant, exhibit, artwork, pet and non-player character assistant.

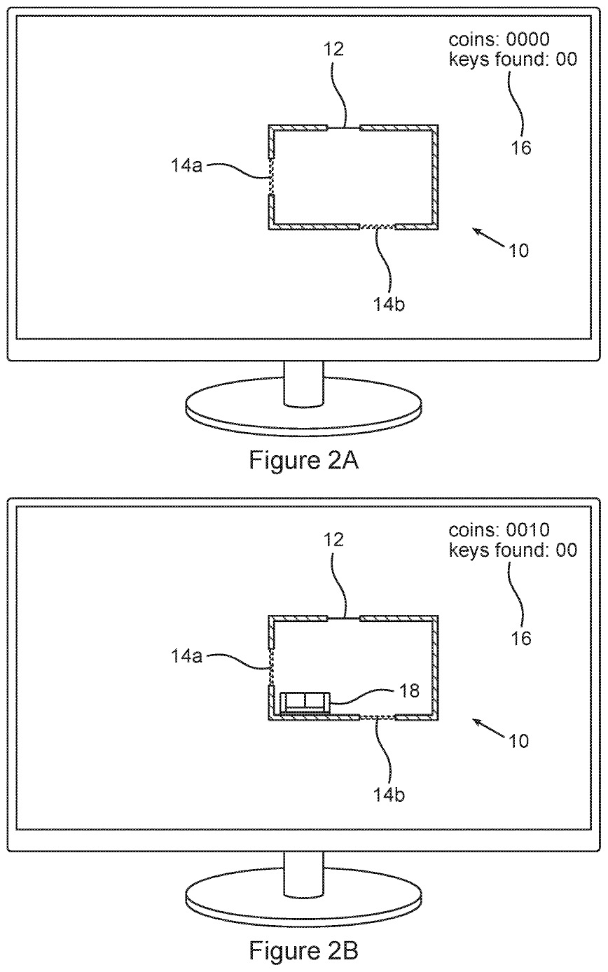

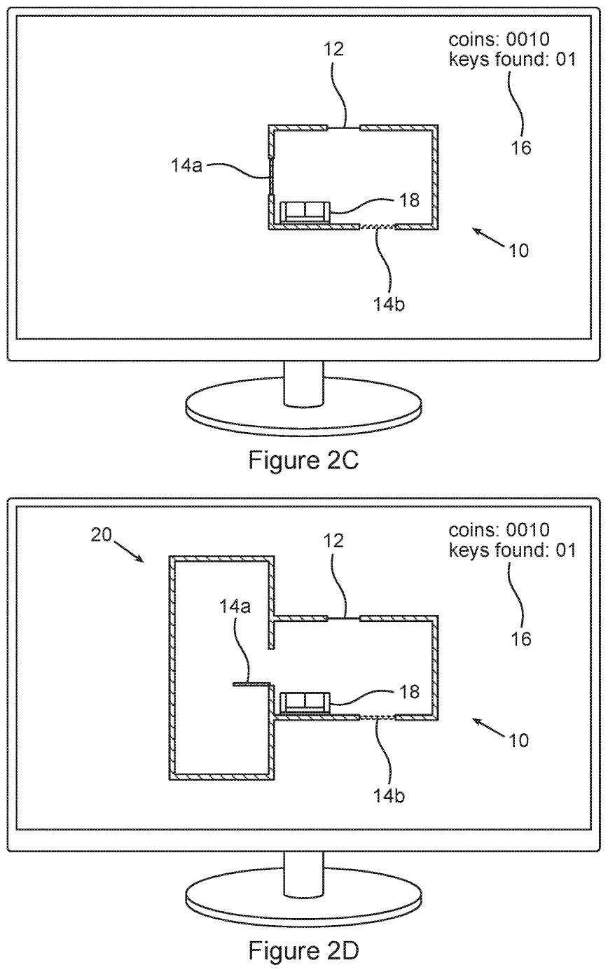

In FIGS. 2A, 2B, 2C and 2D are schematically depicted exemplary screenshots of a display component of an apparatus according to the teachings herein during play of a game. In FIG. 2, the game is a sandbox game where the game base is a virtual room that is part of a virtual house. The rewards are in-game rewards, specifically virtual coins and virtual keys. The coins are used to purchase in-game decorations and furnishings while the keys are used to access otherwise inaccessible portions of the virtual house, each virtual key allowing the opening of a specific virtual door.

FIG. 2A depicts the beginning of the game, where the user has access to a single room 10, which room has a window 12, two doors 14a and 14b. Doors 14a and 14b are displayed in a different color than other parts of room 10, indicating that these are not accessible at this point in the game.

Counter 16 shows the number of coins and keys that the user has received as rewards.

FIG. 2B depicts room 10 after the user has used the wellness device properly for six consecutive days. For each day of use of the wellness device, the user received ten coins, a total of sixty coins. The user purchased a sofa 18 for fifty coins to decorate room 10, and is left with ten coins in counter 16.

FIG. 2C depicts room 10 after the user has used the wellness device properly for seven consecutive days, i.e., one day after the depicted in FIG. 2B. The reward provided was a virtual key as indicated by counter 16, which key can be used to open door 14a as indicated by the change in color of door 14a.

FIG. 2D depicts room 10 after the user used the key to open door 14a, thereby gaining access to an additional virtual space of the virtual house, garden 20.

In an alternative embodiment of the game described with reference to FIGS. 2A, 2B, 2C and 2D, a player receives one or more virtual keys for each correct use of the associated wellness device. The player can use the received keys to open virtual treasure chests, each virtual treasure chest requiring the use of one or more keys to open. Opening such a treasure chest with a key or keys yields in-game rewards (e.g. virtual items to put in room 10) and/or tangible rewards (e.g., coupons) and/or virtual coins. The virtual coins can be used to purchase virtual items to put in room 10 or can be saved. When a player has a sufficient number of items and a sufficient number of keys, the player gains access to an additional room. Access to an additional room is granted only after a sufficient period of time has passed after access to a previous additional room is granted, e.g., a week.

It is believed that at least one advantage of a video game having an in-game base as opposed to games devoid of such an in-game base is that in some instances an in-game base becomes a mnemonic associated with the (correct) use of the wellness device. When the person experiences a need to receive the benefits of using the wellness device but cannot actually use the wellness device, the person plays the game without using the wellness device and consequently automatically recalls the proper activity associated with the use of the wellness device. For example, if the wellness device is a breathing biofeedback device and the user feels a shortness of breath, playing the game helps recall the controlled breathing that is associated with the correct use of the breathing biofeedback device.

1. An apparatus for encouraging a user to use a wellness device, comprising:
a display device having a computer processor functionally associated with a display of the display device, the computer processor configured to:
run at least part of a video game program, and
display, on the display device, game screens to a user playing the video game program,
wherein the display device is further configured to:
receive information from a wellness device about the use of the wellness device; and
provide the received information to the video game program.

2. The apparatus of claim 1, wherein the providing the received information to the video game program is performed when the video game program is being played on display device.

3. The apparatus of claim 1, wherein the providing the received information to the video game program is performed only when the video game program is being played on the display device.

4. The apparatus of claim 1, wherein the providing the received information to the video game program is performed when the video game program is running on the display device.

5. The apparatus of claim 1, wherein when the video game program is not being played on the display device and/or is not running on the display device, the display device is configured to store the received information and to provide the stored information to the video game program at a later time.

6. The apparatus of claim 1, further comprising the wellness device.

7. The apparatus of claim 6, wherein the wellness device is a breathing biofeedback device.

8. The apparatus of claim 1, wherein the video game program is configured to provide rewards for use of the wellness device.

9. (canceled)

10. The apparatus of claim 1, wherein the video game program is configured to provide rewards for correct use of the wellness device.

11. The apparatus of claim 8, wherein the video game program is configured so that a value of the provided rewards is related to a frequency of the use of the wellness device.

12. The apparatus of claim 8, wherein the video game program is configured so that a value of the provided rewards is related to a degree of conformity and/or compliance with a pre-determined schedule for the use of the wellness device.

13. The apparatus of claim 1, wherein the video game program is a game comprising an in-game base.

14. The apparatus of claim 8, wherein at least some of the rewards are in-game rewards.

15. The apparatus of claim 8, wherein at least some of the rewards are tangible rewards.

16. A method for encouraging a user to use a wellness device, comprising:
providing information about the user's use of the wellness device as input for a video game program played by the user on a display device; and
improving, based on the provided information, an in-game experience of the user while playing the video game program.

17. The method of claim 16, wherein the user increases a frequency of the user's use of the wellness device based on the improved in-game experience.

18. The method of claim 16, wherein the user increases a correctness of the user's use of the wellness device based on the improved in-game experience.

19. The method of 17, wherein at least one parameter of the user's well-being increased due to the increased frequency of the user's use of the wellness device.

20. The method of claim 16, wherein the wellness device is a breathing biofeedback device.

21. The method of claim 16, wherein the video game program is a game comprising an in-game base.

22. The method of claim 16, wherein the improving the in-game experience comprises rewarding the user with a reward for using the wellness device more often and/or more correctly compared to one or more previous uses of the wellness device.

23. The method of claim 22, wherein a value of the reward is related to a frequency of the user's use of the wellness device.

24. The method of claim 22, wherein the reward comprises an in-game reward.

25. The method of claim 22, wherein the reward comprises a tangible rewardsreward.

26. The method of claim 18, wherein at least one parameter of the user's well-being is increased due to the increased correctness of the user's use of the wellness device.
